(19) United States
(12) Patent Application Publication
(10) Pub. No.: US 2022/0185509 A1
(43) Pub. Date: Jun. 16, 2022

(54) PROCESSES FOR THE PRODUCTION OF SALINE SOLUTION BAGS

(71) Applicant: Peter Ryan, Midland, MI (US)

(72) Inventor: Peter Ryan, Midland, MI (US)

(21) Appl. No.: 17/122,510

(22) Filed: Dec. 15, 2020

(51) Int. Cl. B65B 3/00; A61J 1/10; A61K 33/14; B65B 55/02; B01D 61/14; B01D 61/20; C02F 1/44; C02F 1/32

(52) U.S. Cl. CPC B65B 3/003; A61J 1/10; A61K 33/14; B65B 55/02; B01D 61/145; C02F 2303/04; C02F 1/444; C02F 1/32; B01D 2311/06; B01D 2311/2619; B01D 2311/2692; B01D 61/20

(57) ABSTRACT

Processes for the production of bags containing saline solution. The processes include sealing bags with crystalline components and then sterilizing the sealed bags with crystalline components. The bags may be sterilized in an autoclave and then transported to a consumption site which produces water-for-injection (WFI) suitable for use in an IV bag. The WFI is added to the sterilized bags, dissolving the solid salt (USP-grade sodium chloride), to produce a bag with a 0.9 w/v % normal saline solution.

### FIELD OF THE INVENTION

The present invention relates to the production of saline solution, and more particularly, to the production of terminally-sterilized bags containing a crystalline ingredient of 0.9% normal saline solution.

### BACKGROUND OF THE INVENTION

Bags of saline solution (i.e., normal saline solution) are used to provide purified water and electrolytes (salts) to a variety of individuals in a hospital or other health care setting. Currently, these bags of saline solution, commonly known as IV bags, are produced at a production location by adding water-for-injection (WFI) and USP-grade salt (NaCl) in a flexible bag. As should be appreciated, “USP-grade” means United States Pharmacopeia (USP) specified produce acceptable for food, drug, or medical use.

More specifically, in conventional processes, WFI is manufactured using city water that is first treated with a reverse-osmosis (RO) membrane followed by a distillation step (vapor-compression or multiple-effect evaporation). The distillate from the distillation step is the WFI product. The WFI is mixed with the USP-grade salt in a sterile mixing vessel to make a 0.9% (w/v) saline solution (9 grams of salt per liter of WFI).

A form-fill-seal (FFS) technology is used form a flexible bag, often Polyvinyl Chloride or Polypropylene (“PVC” or “PP”), fill the flexible bag with the desired amount of the normal saline solution, and seal the flexible bag. The flexible bag product containing the normal saline solution is placed in a rack designed to hold a specific number of flexible bags. The rack is then placed in an autoclave. Typically, several racks filled with flexible bags are placed into the autoclave.

The autoclave provides terminal sterilization of the flexible bags by applying moist heat at 120° C. for 30 minutes. The moist heat is sterile steam (steam made from WFI). The autoclave operates at a pressure of 29 psia to maintain a temperature of 120° C. After terminal sterilization, the bags with a liquid product are packed and prepared for shipment.

While presumably effective for their intended purposes, the conventional process is limited in throughput by the capacity of the autoclave. The conventional saline solution IV bag containing the desired volume of saline solution occupies that amount of volume in the autoclave. For example, if the autoclave has a capacity to terminally sterilize 500 one-liter normal saline solution bags, then this defines the capacity of the autoclave for the conventional process.

Therefore, it would be desirable to provide effective and efficient processes for the production of saline that does not require liquid to be shipped, and that does not occupy as much space when stored or when subject to sterilization.

### SUMMARY OF THE INVENTION

A new process has been invented for the production of saline filled bags that addresses one or more of the aforementioned concerns. More specifically, in the present processes, the bags are filled with solid salt, sealed, and sterilized. The sterilized bags are shipped to an end-user (for example, a hospital or a medical center) which has a system that will convert site-supplied reverse osmosis water into WFI using ultra-filtration and UV sterilization. This allows the bags containing the solid salt to be filled with WFI produced on-site.

The present processes do not include the WFI component of the normal saline solution in the terminal sterilization process. Rather, the present processes only include the crystalline component in the IV bag for terminal sterilization. A liquid-full IV bag occupies approximately four times the volume of a liquid-free IV bag. By omitting the WFI in the terminal sterilization part of the production process, an additional 300% of throughput capacity in the autoclave is realized.

Further, the sterilized bags with solid salt take up less space for storing. Additionally, the cost to ship the bags is lower, and the environmental impact associated with shipping the bags is lessened when the WFI is not included in the bags that are transported.

Accordingly, in an aspect of the present invention, the present invention may be generally characterized as providing a process for producing a saline solution within individual flexible bags by: adding a solid salt to a plurality of flexible bags to provide bags containing the solid salt; sealing the bags containing the solid salt to provide sealed bags; sterilizing the sealed bags to provide sterilized bags; and, adding an aqueous solvent to at least one of the sterilized bags to dissolve the solid salt and provide a bag containing a saline solution.

The aqueous solvent may be water-for-injection. The process may further include a step of manufacturing the water-for-injection by: filtering water from a water source in an ultra-filtration process to provide a filtration effluent; and, sterilizing the filtration effluent to provide the water-for-injection. The filtration effluent may be sterilized by UV sterilization. The water-for-injection may be manufactured in a consumption zone, and wherein the sterilized bags are provided from a production zone. The process may further include shipping one or more of the sterilized bags from the production zone to the consumption zone.

The sealed bags may be sterilized in an autoclave.

The solid salt may include USP grade sodium chloride. The solid salt may further include at least one additive.

The adding of the salt and the sealing of the bags containing solid salt may be performed in a single, sterile compartment of a device.

In another aspect, the present invention may be characterized, broadly, as providing a process for producing a saline solution within individual bags by, in a production zone, adding solid salt to a plurality of bags to provide bags containing solid salt, sealing the bags containing solid salt to provide sealed bags, and, sterilizing the sealed bags to provide sterilized bags, and, in a consumption zone, manufacturing an aqueous solvent comprising water-for-injection, and adding the aqueous solvent to at least one of the sterilized bags to dissolve the solid salt and provide a bag containing a saline solution.

The process may further include transporting at least one of the sterilized bags from the production zone to the consumption zone.

The aqueous solvent may be manufactured by filtering water from a water source in an ultra-filtration process to provide a filtration effluent, and, sterilizing the filtration effluent to provide the water-for-injection. The filtration effluent may be sterilized by UV sterilization.

The solid salt may be USP-grade sodium chloride. The solid salt may further include at least one additive.

The sealed bags may be terminally sterilized in an autoclave.

In a further another aspect, the present invention may be characterized, generally, as providing a process for producing a saline solution within individual bags by: adding solid USP-grade sodium chloride to a plurality of bags to provide bags containing the solid USP-grade sodium chloride; sealing the bags containing the solid USP-grade sodium chloride to provide sealed bags; sterilizing the sealed bags in an autoclave to provide sterilized bags; transporting at least one of the sterilized bags to a consumption zone; manufacturing, in the consumption zone, an aqueous solvent comprising water-for-injection; and, adding, in the consumption zone, the aqueous solvent to at least one of the sterilized bags to dissolve the solid USP-grade sodium chloride and provide a bag containing a saline solution.

The aqueous solvent may be manufactured by filtering water from a water source in an ultra-filtration process to provide a filtration effluent, and, sterilizing the filtration effluent to provide the water-for-injection. Adding solid USP-grade sodium chloride to a plurality of bags and the sealing the bags may be performed in a single, sterile compartment of a device.

These and other aspects and embodiments of the present invention will be appreciated by those of ordinary skill in the art based upon the following description of the drawings and detailed description of the preferred embodiments.

### BRIEF DESCRIPTION OF THE DRAWINGS

The attached drawings will make it possible to understand how the invention can be produced and practiced, in which:

FIG. 1 depicts a process flow diagram of an exemplary embodiment of the present invention.

### DETAILED DESCRIPTION OF THE PREFERRED EMBODIMENTS

As mentioned above, a new process for producing saline IV bags has been invented. Unlike prior processes, the present processes sterilize a solid salt inside of the bag. By sterilizing the bags with solid, as opposed to liquid, more bags may be sterilized in the same space. Additionally, it is believed that less energy is required for sterilizing the bags. The sterilized bags containing the salt may then be sent to the end user for use or consumption (e.g., hospital, clinic, etc.). At the site of consumption, an aqueous solvent is provided to fill the bags and dissolve the salt. The aqueous solvent sterilized and provided as water-for-injection. By shipping just the bags with salt (as opposed to shipping bags with the saline solution) and producing the water onsite, shipping costs may be lowered and the per-bag environmental impact associated with shipping the bags from the production facility to the end user is also reduced.

Accordingly, with reference the attached drawings, one or more embodiments of the present invention will now be described with the understanding that the described embodiments are merely preferred and are not intended to be limiting.

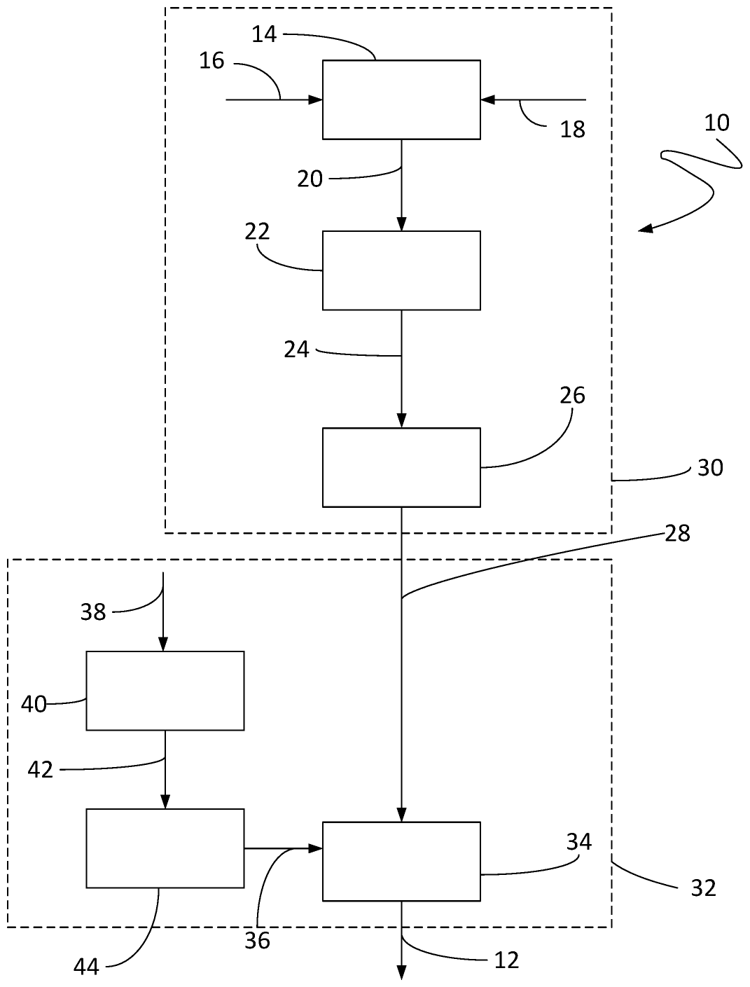

With reference to FIG. 1, an exemplary process 10 for producing bags 12 containing a saline solution is depicted and will be explained with the understanding that variations of the process are contemplated. In a first step 14 of the process 10, solid salt 16 is added to individual bags 18 to provide bags 20 containing the solid salt 16.

The solid salt 16 is preferably a USP-grade salt and most preferably a USP-grade sodium chloride salt. The solid salt 16 may also include at least one additive that is also solid including, for example, nutrients, anesthesia components, freeze-dried therapeutic proteins, etc.

The bags 18 may be made from any suitable material that is typically employed for IV bags. Non-limiting examples of materials for the bags 18 include polymers and copolymers including polypropylene (PP), polyethylene (PE), polyamide (PA), cyclic olefin copolymer (COC), polystyrene (PS), styrene-ethylene-butylene-styrene triblock copolymers (SEBS), styrene-ethylene-propylene-styrene triblock copolymers (SEPS), polyesters, polyvinyl chloride (PVC), ethylene vinyl acetate (EVA), and polychlorotrifluoroethylene (PCTFE), to name a few.

The bags 18 have a size that is selected so that the bags 18 contain a standard volume of saline when filled with an aqueous solvent, such as WFI (discussed below). Typical standard volumes include, 100, 150, 250, 500, and 1000 mL. For a bag 18 with a volume of 1 L, nine grams of solid salt 16 are added to the bag 18.

The bags 18 may include at least one opening that allows for the solid salt 16 to be deposited in the bags 18. The opening may be associated with a seal that is opened to allow the salt 16 to be added into the bag 18.

After the adding step 14, the bags 20 containing the solid salt 16 are subjected to a sealing process 22 to provide sealed bags 24 (that containing solid salt). The sealing process 22 may be any suitable sealing process that seals the bag 18 to at least minimize, and preferably prevent, water vapor and air from entering the bag through the opening through which the salt was added. If the opening is associated with a seal, the sealing process 22 may include activating the seal. Alternatively, the sealing process may include fusing or joining two or more sides of the bag to form the seal by heat or ultra sound, for example.

In a preferred embodiment, the addition 14 of the salt 16 to the bags 18 and the sealing 22 of the bags 20 are performed in a single unit of a form fill seal (FFS) device. In such devices, as are known in the art, the formation of the bag 18, the addition 14 of the salt 16 to the bag 18, and the sealing 22 of the bag 20 containing the salt 16 are performed in a chamber of the device. This chamber is typically sterile and closed to minimize any contamination in the sealed bags 24.

The sealed bags 24 then proceed to a sterilization step 26 which provides sterilized bags 28. In a preferred embodiment, the sterilization step includes placing a plurality of bags in an autoclave, operating at a temperature of 120° C., for 30 minutes. Other sterilization processes, such as UV sterilization, are contemplated.

However, compared with conventional processes, the sealed bags 24 take up less space than conventional bags filled with fluid. Accordingly, this allows for more sealed bags 24 to be placed into the same autoclave than conventionally filled bags (or a different autoclave with the same size). Additionally, without any liquid in the bag, the amount of energy needed to sterilize the sealed bags 24 may be less with the specific energy (energy/unit IV bag) being reduced.

The foregoing steps 14, 22, 26 preferably take place in a production zone 30 that is different from the place the saline solution is used. In other words, the salt 16 is added 14 to the bags 18, the bags 20 are sealed 22, and the sealed bags 24 are sterilized 26 at the production zone 30, such as a factory. The sterilized bags 28 may be transported to a consumption zone 32 wherein the saline solution is used. For example, the consumption zone 32 may be a hospital or a clinic. Accordingly, in some embodiments, the transportation includes shipping the sterilized bags 28 to the consumption zone 32. In some instances, however, the area of production 30 may be in the same building or on the campus location as the consumption zone 32. As noted above, the sterilized bags 28 do not include any liquids, and therefore the costs and environmental impact associated with shipping the sterilized bags 28 is lower compared with conventional processes.

At the consumption zone 32, an aqueous solvent 36 is added 34 to the sterilized bags 28 to dissolve the solid salt and provide the bags 12 containing the saline solution. The aqueous solvent 36 is preferably water-for-injection (WFI) produced at the consumption zone 32.

The water-for-injection may be produced by filtering water from a water source 38 in an ultra-filtration process 40 to provide a permeate, or filtration effluent 42. Preferably the water from the water source 38 is water that has already been subjected to a reverse osmosis treatment. The ultra-filtration process 40 uses pressure to drive the water through a semi-permeable membrane to remove contaminates in the water.

The filtration effluent 42 may then be passed to a sterilization process 44 to provide the water-for-injection 36. Preferably, the sterilization process 44 is a UV sterilization process that passes the filtration effluent 42 through the path of UV light having a wavelength that destroys bacteria, viruses, and other biological material.

A single device may be attached to the water source to perform the ultra-filtration 40, the sterilization 44, and the addition 34 of water. In order to fill the sterilized bags 28 with the aqueous solvent 36, the sterilized bags 28, in a sterile compartment, may be opened, either via a seal or by puncturing the bag. After filled, the bags 28 are resealed or otherwise closed to avoid contamination and provide the bags 12 with the saline solution that may be provided to a consumer or stored.

In addition to the benefits described above, the present processes also allow the consumption zone 32 to store more bags 12 in the same amount of space, and maintain a lower level of inventory of liquid saline solution as the production of same is able to be accomplished on demand. Additionally, the present processes also allow for the sterilized bags 28 to be shipped to remote places which have limited clean water since the present processes provide for on demand water for injection. Additionally, the unfilled saline IV bags may have an unlimited shelf-life.

Furthermore, while the present disclosure has focused on producing bags with a saline solution, it is contemplated that the present processes could be used to produce other sterilized solutions that are produced by dissolving solids in a sterilized bag with solvents, like WFI, that are locally produced. Again, this would allow for improvements in the production and sterilization of the bags, lower shipping costs, and smaller storage requirements.

For example, the bags 12 may be filled with a mixture of sodium chloride, sodium lactate, potassium chloride, and calcium chloride and sterilized. After transportation, the bags 12 may be filled with the aqueous solvent, again preferably WFI, to produce a bag with Lactated Ringer's solution. Similarly, as discussed above, solid nutrients, protein therapeutics, and other crystalline or freeze-dried components may be included the bags and sterilized.

As is apparent from the foregoing specification, the invention is susceptible of being embodied with various alterations and modifications which may differ particularly from those that have been described in the preceding specification and description. It should be understood that we wish to embody within the scope of the patent warranted hereon all such modifications as reasonably and properly come within the scope of this contribution to the art.

1. A process for producing a saline solution within individual flexible bags, the process comprising:
adding a solid salt to a plurality of flexible bags to provide bags containing the solid salt;
sealing the bags containing the solid salt to provide sealed bags;
sterilizing the sealed bags to provide sterilized bags; and,
adding an aqueous solvent to at least one of the sterilized bags to dissolve the solid salt and provide a bag containing a saline solution.

2. The process of claim 1, wherein the aqueous solvent comprises water-for-injection.

3. The process of claim 2, further comprising:
manufacturing the water-for-injection by:
filtering water from a water source in an ultra-filtration process to provide a filtration effluent; and,
sterilizing the filtration effluent to provide the water-for-injection.

4. The process of claim 3, wherein the filtration effluent is sterilized by UV sterilization.

5. The process of claim 3, wherein the water-for-injection is manufactured in a consumption zone, and wherein the sterilized bags are provided from a production zone.

6. The process of claim 5 further comprising:
shipping one or more of the sterilized bags from the production zone to the consumption zone.

7. The process of claim 1, wherein the sealed bags are sterilized in an autoclave.

8. The process of claim 1, wherein the solid salt comprises USP-grade sodium chloride.

9. The process of claim 8, wherein the solid salt further comprises at least one additive.

10. The process of claim 1, wherein the adding of the salt and the sealing of the bags containing solid salt are performed in a single, sterile compartment of a device.

11. A process for producing a saline solution within individual bags, the process comprising:
in a production zone:
adding solid salt to a plurality of bags to provide bags containing solid salt,
sealing the bags containing solid salt to provide sealed bags, and,
sterilizing the sealed bags to provide sterilized bags; and,
in a consumption zone:
manufacturing an aqueous solvent comprising water-for-injection, and
adding the aqueous solvent to at least one of the sterilized bags to dissolve the solid salt and provide a bag containing a saline solution.

12. The process of claim 11 further comprising:
transporting the at least one of the sterilized bags from the production zone to the consumption zone.

13. The process of claim 11, wherein the aqueous solvent is manufactured by:
filtering water from a water source in an ultra-filtration process to provide a filtration effluent; and,
sterilizing the filtration effluent to provide the water-for-injection.

14. The process of claim 13, wherein the filtration effluent is sterilized by UV sterilization.

15. The process of claim 11, wherein the solid salt comprises USP-grade sodium chloride.

16. The process of claim 15, wherein the solid salt further comprises at least one additive.

17. The process of claim 11, wherein the sealed bags are sterilized in an autoclave.

18. A process for producing a saline solution within individual bags, the process comprising:
adding solid USP-grade sodium chloride to a plurality of bags to provide bags containing the solid USP-grade sodium chloride;
sealing the bags containing the solid USP-grade sodium chloride to provide sealed bags;
sterilizing the sealed bags in an autoclave to provide sterilized bags;
transporting the at least one of the sterilized bags to a consumption zone;
manufacturing, in the consumption zone, an aqueous solvent comprising water-for-injection; and,
adding, in the consumption zone, the aqueous solvent to the at least one of the sterilized bags to dissolve the solid USP-grade sodium chloride and provide a bag containing a saline solution.

19. The process of claim 18, wherein the aqueous solvent is manufactured by:
filter water from a water source in an ultra-filtration process to provide a filtration effluent; and,
sterilizing the filtration effluent to provide the water-for-injection.

20. The process of claim 19, wherein the adding solid USP-grade sodium chloride to a plurality of bags and the sealing the bags are performed in a single, sterile compartment of a device.
